(19) United States
(12) Patent Application Publication
(10) Pub. No.: US 2025/0066788 A1
(43) Pub. Date: Feb. 27, 2025

(54) USE OF CCL7 ANTAGONISTS FOR THE PREVENTION OR TREATMENT OF DIABETIC KIDNEY DISEASE

(71) Applicant: National Yang Ming Chiao Tung University, Hsinchu County (TW)

(72) Inventors: Ting-Ting Chang, Hsinchu County (TW); Jaw-Wen Chen, Hsinchu County (TW)

(73) Assignee: National Yang Ming Chiao Tung University, Hsinchu County (TW)

(21) Appl. No.: 18/649,626

(22) Filed: Apr. 29, 2024

(30) Foreign Application Priority Data
Aug. 24, 2023 (TW) 112131921

(51) Int. Cl. C12N 15/113; A61P 13/12; C07K 16/24; C07K 16/28

(52) U.S. Cl. CPC C12N 15/1136; A61P 13/12; C07K 16/24; C07K 16/2866; C12N 15/1138; C07K 2317/76; C12N 2310/14; C12N 2310/16

(57) ABSTRACT

Provided are compositions and methods of use of a pharmaceutical composition in the manufacture of a medicament for preventing or treating a diabetic kidney disease, the pharmaceutical composition including a chemokine C-C motif ligand 7 (CCL7) antagonist and/or a pharmaceutically acceptable salt thereof and a pharmaceutically acceptable carrier. The medicament can prevent or treat diabetic kidney disease by protecting tubular epithelial cells, reducing glomerular hypertrophy, glomerulosclerosis, and fibrosis. The present disclosure also provides a method for preventing or treating a diabetic kidney disease in a subject in need thereof, including administering an effective amount of a CCL7 antagonist and/or a pharmaceutically acceptable salt thereof to the subject to inhibit an activity of CCL7.

### CROSS-REFERENCE TO RELATED APPLICATION

This application claims the benefit of priority of Taiwan Patent Application No. 112131921, filed Aug. 24, 2023, the entire contents of which are hereby incorporated by reference.

### BACKGROUND

### Technical Field

The present disclosure relates to the prevention, treatment, and improvement of a diabetic kidney disease. More particularly, the disclosure relates a method and use for the prevention, treatment, and improvement of a diabetic kidney disease with a chemokine C-C motif ligand 7 (CCL7) antagonist.

### Description of Related Art

Diabetic kidney disease (DKD), a progressive kidney disease, is characterized by proteinuria, glomerular and kidney hypertrophy, accumulation of extracellular matrix in glomeruli, kidney inflammation/fibrosis, and progressive decline in renal function. Diabetic kidney disease has been increasingly prevalent with the increase of obesity and the incidence rate of type 2 diabetes. As reported by the International Diabetes Association, there are 425 million people with diabetes worldwide in 2019, and this number is expected to reach 629 million by 2045, and it is anticipated that about 40% of these diabetic individuals will develop diabetic kidney disease.

Currently, the clinical medication used to treat diabetic kidney diseases predominantly includes the angiotensin-converting-enzyme (ACE) inhibitors and the angiotensin II receptor antagonists, which reduce proteinuria by mechanisms including reducing the high pressure in the renal glomerulus and regulating the proliferation of renal glomerulus microvessels. However, only treating proteinuria cannot reverse the progression of diabetic kidney disease and has no direct and clear causal relationship with long-term renal function prognosis. In fact, there is still a considerable proportion of patients that require hemodialysis due to progressive renal insufficiency after treatment every year. According to statistics, about 45% of the new hemodialysis patients in China are caused by diabetic kidney disease. Therefore, there is an urgent unmet need for new inventive medicaments for preventing and/or treating the diabetic kidney disease.

### SUMMARY

In view of the foregoing, the present disclosure provides a method for preventing or treating a diabetic kidney disease in a subject in need thereof, comprising administering an effective amount of a CCL7 antagonist and/or a pharmaceutically acceptable salt thereof to the subject to inhibit an activity of CCL7.

In at least one embodiment of the present disclosure, the CCL7 antagonist is an antibody or an aptamer against the CCL7 or a CCL7 receptor.

In at least one embodiment of the present disclosure, the CCL7 antagonist is at least one selected from the group consisting of a CCL7 neutralizing antibody, a CCL7 RNA interference agent, a CCL7 small molecular antagonist, a C-C chemokine receptor type 1 (CCR1) antagonist, a C-C chemokine receptor type 2 (CCR2) antagonist, a C-C chemokine receptor type 3 (CCR3) antagonist, and a C-C chemokine receptor type 5 (CCR5) antagonist.

In at least one embodiment of the present disclosure, the CCL7 antagonist is the CCL7 neutralizing antibody and/or the CCL7 RNA interference agent.

In at least one embodiment of the present disclosure, the administering protects renal functions, reduces proteinuria, reduces kidney hypertrophy, reduces kidney inflammation and/or reduces kidney fibrosis in the subject.

In at least one embodiment of the present disclosure, the CCL7 antagonist and/or a pharmaceutically acceptable salt thereof is administered orally, intraperitoneally, intravenously, intradermally, intramuscularly, subcutaneously, or transcutaneously.

In at least one embodiment of the present disclosure, the CCL7 antagonist and/or a pharmaceutically acceptable salt thereof is administered in combination with an angiotensin-converting-enzyme inhibitor and/or an angiotensin II receptor antagonist.

In at least one embodiment of the present disclosure, the subject has urinary albumin.

In at least one embodiment of the present disclosure, the subject has an increase in an expression of an inflammatory factor and/or a fibrous protein in kidney, an increase in a level of a serum blood urea nitrogen, an increase in a level of a serum creatinine, an increase in a kidney-to-body weight ratio, and/or an increase in a urinary albumin-creatinine ratio.

In at least one embodiment of the present disclosure, the diabetic kidney disease is caused by at least one disorder of diabetes, hypertension, and hyperlipidemia.

In at least one embodiment of the present disclosure, the diabetes is type 1 diabetes or type 2 diabetes.

In at least one embodiment of the present disclosure, the subject is a human or an animal.

In at least one embodiment of the present disclosure, the effective amount of the CCL7 antagonist and/or a pharmaceutically acceptable salt thereof is about 0.01 μg/kg to about 100 mg/kg.

In at least one embodiment of the present disclosure, the effective amount of the CCL7 antagonist and/or a pharmaceutically acceptable salt thereof is about 0.1 μg/kg to about 1 mg/kg.

In at least one embodiment of the present disclosure, the CCL7 antagonist is administered orally, intraperitoneally, intravenously, intradermally, intramuscularly, subcutaneously or transdermally.

In at least one embodiment of the present disclosure, the method further comprises administering an angiotensin-converting-enzyme (ACE) inhibitor and/or an angiotensin II receptor antagonist to the subject.

Further, the present disclosure provides the use of a pharmaceutical composition in manufacture of a medicament for preventing or treating a diabetic kidney disease, and the pharmaceutical composition comprises a CCL7 antagonist and/or a pharmaceutically acceptable salt thereof, and a pharmaceutically acceptable carrier.

In at least one embodiment of the present disclosure, the CCL7 antagonist is an antibody or an aptamer against the CCL7 or a CCL7 receptor.

In at least one embodiment of the present disclosure, the CCL7 antagonist is at least one selected from the group consisting of a CCL7 neutralizing antibody, a CCL7 RNA interference agent, a CCL7 small molecular antagonist, a CCR1 antagonist, a CCR2 antagonist, a CCR3 antagonist, and a CCR5 antagonist.

In at least one embodiment of the present disclosure, the CCL7 antagonist is the CCL7 neutralizing antibody and/or the CCL7 RNA interference agent.

In at least one embodiment of the present disclosure, the medicament protects renal functions, reduces proteinuria, reduces kidney hypertrophy, reduces kidney inflammation and/or reduces kidney fibrosis.

In at least one embodiment of the present disclosure, the medicament is administered orally, intraperitoneally, intravenously, intradermally, intramuscularly, subcutaneously, or transcutaneously.

In at least one embodiment of the present disclosure, the medicament is administered in combination with an ACE inhibitor and/or an angiotensin II receptor antagonist, or the pharmaceutical composition includes the ACE inhibitor and/or the angiotensin II receptor antagonist.

In at least one embodiment of the present disclosure, the diabetic kidney disease is accompanied with urinary albumin.

In at least one embodiment of the present disclosure, the diabetic kidney disease is caused by at least one disorder of diabetes, hypertension, and hyperlipidemia.

In at least one embodiment of the present disclosure, the diabetes is type 1 diabetes or type 2 diabetes.

In at least one embodiment of the present disclosure, the diabetic kidney disease includes an increase in an expression of an inflammatory factor and/or a fibrous protein in kidney, an increase in a level of a serum blood urea nitrogen, an increase in a level of a serum creatinine, an increase in a kidney-to-body weight ratio, and/or an increase in a urinary albumin-creatinine ratio.

In at least one embodiment of the present disclosure, the CCL7 antagonist and/or the pharmaceutically acceptable salt thereof is present at an amount of 0.01 to 80 wt % in the pharmaceutical composition based on a total weight of the pharmaceutical composition.

According to the present disclosure, the CCL7 antagonist can reduce the expression of inflammatory and fibrosis factors such as TNF-α, IL-6, IL-1β and TGF-β in renal tubular epithelial cells in a high glucose environment and further suppress CCL7 in diabetes patients, so as to protect a renal function, reduce levels of serum blood urea nitrogen (BUN) and serum creatinine and to reduce the kidney-to-body weight ratio, and meanwhile to improve the urinary albumin-creatinine ratio, thereby protecting the overall renal function, reducing proteinuria, reducing kidney hypertrophy, reducing kidney inflammation, and/or reducing kidney fibrosis, and thus preventing and/or treating the diabetic kidney disease.

As used in the present disclosure, the singular forms “a,” “an” and “the” include a plurality of the referents unless explicitly and unequivocally limited to one referent. The terms “or” and “and/or” can be used interchangeably unless the context clearly indicates otherwise.

As used herein, the term “include” or “comprise” refers to the composition, method and individual component(s) thereof essential to the present disclosure, yet open to the unspecified elements, regardless of essential or not.

The terms “subject” and “patient” are used interchangeably in the present disclosure, and the term “subject” refers to a human or an animal. Examples of the subject includes, but not limited to, human, monkey, mouse, rat, marmot, ferret, rabbit, hamster, cattle, horse, pig, deer, dog, cat, fox, wolf, chicken, emu, ostrich and fish. In certain embodiments of the present disclosure, the subject is a mammal, e.g., a primate, such as a human.

The term “treatment” intends to include alleviating or eliminating a disorder, disease or condition, or one or more symptom(s) associated with the disorder, disease or condition; or alleviating or eradicating the underlying cause of the disorder, disease or condition.

The term “prevention” means to include a method of delaying and/or preventing a disorder, disease or condition and/or the onset of accompany symptoms thereof; protecting a subject from the disorder, disease or condition; or reducing the risk of developing the disorder, disease or condition.

The term “therapeutic effective amount” or “effective amount” means an amount of an active ingredient (e.g., a CCL7 antagonist) administered to a subject, which is sufficient to prevent or treat the development of a disorder, disease or condition or to alleviate the disorder, disease or condition to an extent. As known by one of ordinary skill in the art, an effective amount can vary depending on the administering route, the excipient used, the other components co-administered, and the condition to be treated.

The value ranges used herein are inclusive and combinable, and any value falling into the value ranges herein can be used as the maximum or the minimum value to derive a subrange therefrom. For example, the value range of “0.01 mg to 1000 mg” should be interpreted to include any subrange between the minimum value of 0.01 mg to the maximum value of 1000 mg, e.g., subranges such as from 0.01 mg to 800 mg, from 0.5 mg to about 700 mg, from 1 mg to about 600 mg, etc. Additionally, a plurality of values herein can be selectively chosen as the maximum and the minimum values of the derived subranges. For example, a value range of 0.01 mg to 5 mg, 0.01 mg to 30 mg, and 5 mg to 30 mg can be derived from the values 0.01 mg, 5 mg and 30 mg.

The term “about” or “approximate” refers to an acceptable error of a specific value determined by one of ordinary skill in the art, which is depended on how the value is measured or determined. In some embodiments, the term “about” or “approximate” refers to be within a range of 20%, 15%, 10%, 9%, 8%, 7%, 6%, 5%, 4%, 3%, 2%, 1%, 0.5%, or 0.05% from a given value.

The method of the present disclosure is to prevent or treat a diabetic kidney disease in a subject in need thereof by a CCL7 antagonist and/or a pharmaceutically acceptable salt thereof. The CCL7 antagonist refers to any antibody or aptamer which is capable of inhibiting CCL7 activity and is against CCL7 or CCL7 receptor. For example, but not limited to, a CCL7 neutralizing antibody, a CCL7 RNA interference agent, a CCL7 small molecular antagonist, a CCR1 antagonist, a CCR2 antagonist, a CCR3 antagonist and a CCR5 antagonist. In one embodiment of the present disclosure, the CCL7 antagonist is the CCL7 neutralizing antibody and/or the CCL7 RNA interference agent. In an embodiment of the present disclosure, the CCL7 antagonist can inhibit the binding of CCL7 to its receptor. In another embodiment of the present disclosure, the CCL7 antagonist can inhibit intracellular signaling generated by the binding of CCL7 to its receptor. For example, the CCL7 antagonist can act on at least one receptor of the CCL7 and CCL7 receptor, thereby blocking the CCL7 signaling. As used herein, the CCL7 receptor includes, but not limited to, CCR1, CCR2, CCR3, and CCR5.

As used herein, the term “aptamer” refers to a class of molecules representing antibody alternatives in molecule recognition. An aptamer is an oligonucleotide or an oligopeptide sequence which has the ability to recognize a target molecule of actually any type with a high affinity and specificity.

As used herein, “CCR1” or “CCR1 receptor,” “CCR2” or “CCR2 receptor,” “CCR3” or “CCR3 receptor,” and “CCR5” or “CCR5 receptor” can be used interchangeably and have the general meanings thereof in the art. CCR1, CCR2, CCR3 and CCR5 receptors can be derived from any source, but generally are CCR1, CCR2, CCR3 and CCR5 receptors derived from mammals (e.g., human or non-human primates). In some embodiments of the present disclosure, the CCR1, CCR2, CCR3, and CCR5 receptors are human receptors.

As used herein, the term “CCL7” has the general meaning in the art. CCL7 is the ligand of CCR1, CCR2, CCR3, and CCR5 receptors and can be derived from any source, but generally is a mammal (e.g., human or non-human primates) CCL7. In on embodiment of the present disclosure, CCL7 is a human CCL7.

As used herein, the term “diabetic kidney disease” refers to a nephropathy induced by uncontrolled high blood glucose, hypertension and/or hyperlipidemia in a diabetes patient (including a patient with type 1 diabetes and type 2 diabetes). In general, the diabetic kidney disease can be diagnosed by measurement of urinary albumin in urine and/or renal functions (for example, estimated glomerular filtration rate (eGFR)). The normal value of urinary albumin-creatinine ratio (UACR) is less than 30 mg/g. when the UACR value is between 30 and 300 mg/g, which is referred to as “microalbumin,” there has been considerable impairment/lesion in kidney, and the patient can have diseases of high blood pressure, abnormal blood lipid, and diseases including neural and fundus. If the situation continues to worsen, when the measured UACR value is greater than 300 mg/g, which is referred to as “macroalbumin,” the renal function has been severely damaged/diseased, and the blood pressure and lipid in the patient have been abnormally aggravated. More severe retinal, neurological, and large vessel lesions are often accompanied, and there is eventually a high probability of hemodialysis at this stage.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a graph showing the distribution of concentrations of plasma CCL7 of the “control” group (non-diabetes subjects), the “UACR <30” group (diabetes subjects having urinary albumin-creatinine ratio of <30 mg/g), the “UACR 30-300” group (diabetes subjects having urinary albumin-creatinine ratio between 30 and 300 mg/g) and the “UACR >300” group (diabetes subjects having urinary albumin-creatinine ratio of >300 mg/g), with the plasma CCL7 concentration in the diabetes subjects being higher than that in the control group of non-diabetes subjects (n=8 per group). *P<0.05, **P<0.01.

FIGS. 2A and 2B are graphs showing the Western blots and quantification of expression of inflammatory factors including CCL7, TNF-α, IL-6, IL-1β, TGF-β as well as fibrosis protein and actin in human proximal renal tubular epithelial cells in the “control” group, the “HG” group (high glucose treatment), the “HG+siC” group (administration of siRNA control followed by high glucose treatment) and the “HG+siCCL7” group (administration of CCL7 siRNA followed by high glucose treatment). The expression level of CCL7 reduced (FIG. 2A; n=3), and the expression levels of TNF-α, IL-6, IL-1β and TGF-β also reduced (FIG. 2B; n=3) in the renal tubular epithelial cells following high glucose treatment after knockdown of gene expression by administering CCL7 siRNA. *P<0.05, **P<0.01.

FIGS. 3A-3H are graphs showing the distribution of blood glucoses (FIG. 3A; n=6), water intakes (FIG. 3B; n=6), body weights (FIG. 3C; n=6), serum BUN levels (FIG. 3D; n=6), serum creatinine levels (FIG. 3E; n=6), serum uric acid levels (FIG. 3F; n=6), kidney-to-body weight ratios (FIG. 3G; n=6) and urinary albumin-creatinine ratios (FIG. 3H; n=6) in the “control” group (non-diabetes mice), the “DKD” group (mice having diabetic kidney disease), the “DKD+CCL7 Ab 0.1 μg” group (mice having diabetic kidney disease treated with 0.1 μg of CCL7 antibody), the “DKD+CCL7 Ab 1 μg” group (mice having diabetic kidney disease treated with 1 μg of CCL7 antibody), the “DKD+IgG Ab 1 μg” group (mice having diabetic kidney disease treated with 1 μg of IgG antibody). The mice having diabetic kidney disease exhibited reductions in water intakes, serum BUN levels, serum creatinine levels, kidney-to-body weight ratios, and urinary albumin-creatinine ratios after the treatment with CCL7 antibodies, suggesting that the CCL7 has the function of renal protection. *P<0.05, **P<0.01.

FIGS. 4A-4C are graphs showing the HE staining of the representative kidney sections and the distribution of glomerular areas (FIG. 4A; n=6), the PAS staining of the representative kidney sections and the distribution of glomerulosclerosis scores (FIG. 4B; n=6), the Masson's trichrome staining of the representative kidney sections and the distribution of Masson's positive areas (FIG. 4C; n=6) in the “control” group (non-diabetes mice), the “DKD” group (mice with diabetic kidney disease), the “DKD+CCL7 Ab 0.1 μg” group (mice having diabetic kidney disease treated with 0.1 μg of CCL7 antibody) and the “DKD+CCL7 Ab 1 μg” group (mice with diabetic kidney disease treated with 1 μg of CCL7 antibody) The mice with diabetic kidney disease exhibit reduced glomerular areas, reduced glomerulosclerosis score, and reduced Masson's positive areas after the treatment with CCL7 antibody, suggesting that the CCL7 antibody can prevent and/or treat glomerular hypertrophy, glomerular sclerosis and/or glomerular fibrosis in the mice of diabetic kidney disease. *P<0.05, **P<0.01.

### DETAILED DESCRIPTION

The following examples are used to illustrate the present disclosure. One with ordinary skill in the art can easily conceive of the other advantages and effects of the present disclosure based on the invention of the specification. The present disclosure can also be implemented or applied as described in various examples. It is possible to modify or alter the following examples for carrying out the present disclosure without violating its spirit and scope, for different aspects and applications.

Chemokines, a family of cytokines, are small molecular proteins secreted by cells, which have the activity of inducing chemotaxis and can mediate the migration and location of immune cells. Chemokines can be classified into several subfamilies, among which CCL7 is of the C-C subfamily, it has been known to be associated with the development, invasion, and metastasis of a tumor and to be involved in the inflammatory reaction by attracting the immune cells. However, the role of chemokines including CCL7 in the diabetic kidney disease remains unknown.

The present disclosure is based, at least in part, on the unexpected discovery that that inhibition of CCL7 effectively suppresses the expression of the inflammatory factors and/or fibrous protein in epithelial cells, thereby: reducing the dysphoric symptom in mice having diabetic kidney disease; ameliorating each biochemical value, reducing the kidney-to-body weight ratio; and decreasing glomerular hypertrophy, glomerulosclerosis and fibrosis, which thereby protect the renal functions, achieving the effect of preventing and/or treating diabetic kidney disease. Accordingly, the techniques herein provide that the administration of any CCL7 antagonist (e.g., any antibody, RNA agent, small molecular protein, compound, product from human body, natural product, natural extract, and the like, capable of inhibiting the CCL7 activity) to a subject or patient can achieve an inhibitory effect on CCL7, thereby preventing and/or treating diabetic kidney disease.

Even though numerous characteristics and advantages of the present disclosure have been set forth in the foregoing description, together with details of the structure and features of the invention, the invention is illustrative only. Changes may be made in the details, especially in matters of shape, size, and arrangement of parts within the principles of the invention to the full extent indicated by the broad general meaning of the terms in which the appended claims are expressed.

The techniques herein provide methods for preventing or treating a diabetic kidney disease in a subject in need thereof, comprising administering an effective amount of a CCL7 antagonist and/or a pharmaceutically acceptable salt thereof to the subject. By administration of the CCL7 antagonist and/or the pharmaceutically acceptable salt thereof, the method reduces the expression of inflammatory and fibrosis factors (wherein the kidney refers to for example renal tubular epithelial cells; the inflammatory factors refer to for example cytokines and/or chemokines, e.g. TNF-α, IL-6, IL-1β and TGF-β) in a high glucose environment, and further suppress CCL7 in a patient with diabetes to protect the renal function, to reduce levels of serum blood urea nitrogen and creatinine and to reduce the kidney-to-body weight ratio, and meanwhile to improve the urinary albumin-creatinine ratio, thereby protecting the overall renal function, reducing proteinuria, reducing kidney hypertrophy, reducing kidney inflammation, and/or reducing kidney fibrosis, and thus achieving the effects of preventing and/or treating a diabetic kidney disease.

The techniques herein provide that the CCL7 antagonist and/or the pharmaceutically acceptable salt thereof may be administered to a subject systemically or locally. In some embodiments, the CCL7 antagonist and/or the pharmaceutically acceptable salt thereof is administered to a subject systemically to reduce the damage to cells all over the body caused by the high glucose environment of diabetes. In some embodiments, the CCL7 antagonist and/or the pharmaceutically acceptable salt thereof is administered to the kidney of a subject locally. In some embodiments, the CCL7 antagonist and/or the pharmaceutically acceptable salt thereof is administered to a selected region of the kidney of a subject locally to prevent and/or treat a diabetic kidney disease in the target body site of the subject.

In some embodiments of the present disclosure, the medicament or method provided in the present disclosure has no limit on the age or health of the target subjects, but may be preferred for use in a subject having diabetes or a diabetic kidney disease.

In some embodiments of the present disclosure, the CCL7 antagonist or the salt thereof is present in the pharmaceutical composition at an amount of about 0.01 to about 80 wt %, such as about 0.01 to about 60 wt %, about 0.01 to about 30 wt %, about 0.01 to about 15 wt %, about 0.1 wt % to about 60 wt %, about 0.1 wt % to about 40 wt %, about 0.1 wt % to about 20 wt %, about 0.5 wt % to about 70 wt %, about 0.5 wt % to about 50 wt %, about 0.5 wt % to about 30 wt %, about 1 wt % to about 75 wt %, about 1 wt % to about 60 wt %, about 1 wt % to about 45 wt %, about 5 wt % to about 50 wt %, about 10 wt % to about 40 wt %, or about 15 wt % to about 30 wt %, based on the total weight of the pharmaceutical composition. In another embodiment, the effective amount of a CCL7 antagonist has a lower limit selected from 0.01 μg/kg, 0.05 μg/kg, 0.1 μg/kg, 0.5 μg/kg, 1 μg/kg, 5 μg/kg, 10 μg/kg, 0.01 mg/kg, 0.05 mg/kg, 0.1 mg/kg, 0.5 mg/kg, 1 mg/kg, 5 mg/kg, 10 mg/kg, 15 mg/kg, 20 mg/kg and 25 mg/kg, and an upper limit selected from 1000 mg/kg, 900 mg/kg, 800 mg/kg, 700 mg/kg, 600 mg/kg, 500 mg/kg, 400 mg/kg, 300 mg/kg, 200 mg/kg, 100 mg/kg, 90 mg/kg, 80 mg/kg, 70 mg/kg, 60 mg/kg, 50 mg/kg, 40 mg/kg and 30 mg/kg.

The techniques herein provide that the CCL7 antagonist is used at a frequency of once to 3 times every day. In another embodiment of the present disclosure, the CCL7 antagonist is used at a frequency of once to 15 times every week. For example, the CCL7 antagonist is administered twice every day or twice every week. In still another embodiment, the CCL7 antagonist is administered once every week, or twice every week, or once every 3 weeks, or once every 4 weeks.

As used herein, the term “administration” refers to applying an active ingredient (e.g., a CCL7 antagonist and/or a salt thereof) by a method or route resulting in that the active ingredient locates at least at a desirable site to generate a desirable effect. The active ingredient provided in the present disclosure can be administered by any suitable route known in the art, including but not limited to, oral, intraperitoneal, intravenous, intradermal, intramuscular, subcutaneous or transdermal manner.

The pharmaceutical composition provided in the present disclosure can be formulated into any dosage form suitable for local administration to achieve a local or systematic effect, including tablets, pills, granules, sublingual tablets, film coated tablets, injections, emulsions, solutions, suspensions, creams, gels, hydrogels, ointments, powders, dressings, elixirs, lotions, suspensions, tinctures, pastes, foams, thin films, aerogels, irrigations, sprays, suppositories, bandages and skin patches.

The pharmaceutically acceptable carriers suitable for the local formulations provided in the present disclosure include but not limited to aqueous carriers, water miscible carriers, non-aqueous carriers, antimicrobials, preservatives, stabilizers, dissolution enhancers, isotonic agents, buffers, antioxidants, local anesthetics, suspending agents, dispersing agents, wetting agents, emulating agents, complexing agents, chelating agents, penetration enhancers, cryoprotectants, freeze-drying protecting agents, thickeners and inert gases.

The pharmaceutical composition provided in the present disclosure can also be administered locally by electroporation, iontophoresis, sonic induction, ultrasonic induction, microneedle or needleless injection, e.g., PowderTect (Chiron Corp., Emeryville, CA) and Bioject (Bioject Medical Technologies Inc., Tualatin, OR).

The form of pharmaceutical composition suitable for injection includes a sterilized aqueous solution or dispersion and sterilized powders for temporary preparation of sterilized injectable solution or dispersion. In all instances, the form must be sterilized and must be a fluid in the extent of easy injection. It must be stable under the conditions for manufacture and storage, and must prevent the pollution caused by microorganism such as bacteria and fungi. The carrier can be a solvent or a dispersing medium comprising, e.g., water, ethanol, polyol (e.g., glycerol, propylene glycol or liquid polyethylene glycol, etc.), and suitable mixture thereof, as well as a vegetable oil. Optionally, various antibacterial agent and antifungal agent, for example, parabens, chlorobutanol, phenol, sorbic acid, merthiolate sodium, etc. In many cases, isotonic agent such as a sugar or sodium chloride preferably. The absorption duration of the injected composition can be prolonged by using an absorption delaying agent in the composition, for example, aluminum monostearate and gelatin.

To meet the requirement of clinical use, the pharmaceutical composition provided in the present disclosure also can be used in combination with other drugs contributed to the prophylaxis and/or treatment of diabetic kidney disease medicament, the drugs include, for example, but not limited to, diabetic kidney disease therapeutic drugs (e.g., ACE inhibitors, angiotensin II receptor antagonists, calcium ion blocking agents), hypoglycemic agents (e.g., sulfonylurea drugs, biguanide drugs, inhibitors of gastrointestinal carbohydrate-digesting enzymes, insulin sensitizing agents), antihypertensives (e.g., diuretics, β1-receptor blocking agents), and/or antilipemic agents (e.g., statin drugs, fibric-acid derivatives).

### EXAMPLES

The efficacies of the present disclosure will be further illustrated in the Examples below which are not intended to limit the scope of the present disclosure.

### Example 1: Materials and Methods

1. Cell Culture and Transfection of CCL7 siRNA

Human renal proximal tubular epithelial cells (HK-2 cells, obtained from ATCC (ATCC, CRL-2190)) were cultured in a DMEM containing 10% fetal bovine serum and 1.0% Penicillin-Streptomycin solution at 37° C. under an atmosphere of 5% CO₂ and 95% air. In the group mimicking the condition of hyperglycemia in a diabetes patient, the cells were cultured with 25 mM glucose for 2 days. In the groups inhibiting CCL7 siRNA (Santa Cruz Biotechnologies, sc-72035) or a siRNA control (Santa Cruz Biotechnologies, sc-37007), Lipofectamine 2000 (Invitrogen) was used in the culture medium, and the cells were transfected with CCL7 siRNA (Santa Cruz) or the siRNA control (Santa Cruz) and cultured with 25 mM glucose.

### 2. Protein Extraction of Proteins and Western Blotting

The protein at equal quantity were subjected to SDS-PAGE (Bio-Rad Laboratories) using a gradient gel of 4-12% under a reduction condition and transferred to a nitrocellulose membrane (GE Healthcare). The membrane was co-cultured with antibodies against CCL7 (R&D Systems), IL-1β, IL-6, TNF-α (Santa Cruz), and TGF-β1 (Cell Signaling Technology). The immunoblotting expression levels of the proteins described above were normalized to the expression level of actin, wherein the expression level of actin was determined by using a mice monoclonal anti-actin antibody (Merck).

### 3. Preparation of the Animal Model

The 6-week old BKS.Cg-Dock7^{m}+/+ Lepr^{db} mice and their wild-type littermates were purchased from the National Laboratory Animal Center (Taipei, Taiwan). The animals were randomized to a control group, a group injected with the CCL7 neutralizing antibody (0.1 or 1 μg of Ab, intraperitoneal injection; R&D Systems; AF-456), and a group injected with the IgG antibody (1 μg of Ab, intraperitoneal injection; R&D Systems; AB-108-C). The neutralizing antibody and IgG were injected 3 times per week for 4 weeks. The mice were raised under conditions without specific pathogens, and all mice were raised in micro isolation cages in accordance with the regulations of the Animal Care Committee of National Yang Ming Chiao Tung University, in a 12-hour day and night cycle at the animal center.

### 4. Biochemistry of Blood and Urine

The blood glucose was measured by using an Abbott free style glucometer (Abbot-OPTIUM XCEED). The levels of the serum blood urea nitrogen (BUN), serum creatinine (Cre) and serum uric acid were measured by using an autoanalyzer (Dri Chem NX500i; FUJIFILM). The mice were placed in metabolism cages for collection of urine over 24 hours, and levels of urinary albumin and creatinine were measured by an ELISA kit (Exocell) in accordance with the instruction of the manufacturer.

### 5. Measurement of the Change in Histopathology

The severity of the diabetic kidney disease was estimated by the glomerular hypertrophy, kidney-to-body weight ratios, and proteinuria. The sections taken from each mouse were stained by the hematoxylin and eosin (HE) staining. Six mice were selected from each group and 20 glomeruli were taken from each mouse to measure the glomerular volume which was calculated by serial sections. The histological changes were quantified to the sum of pixel values in each glomerular area using the ImageJ 1.44 version image analysis software.

### 6. Periodic Acid-Schiff (PAS) Staining and Glomerulosclerosis Score

The kidney taken from a mouse was fixed with 10% paraformaldehyde, dehydrated, and embedded in paraffin. It was sliced into sections of 3 μm thick and was stained with 0.5% periodic acid solution for 5 minutes. The sections were rinsed with distilled water and stained with a Schiff reagent for 15 minutes. Finally, the sections were washed with distilled water for 5 minutes and counterstained with Mayer hematoxylin for 1 minute. Images of the sections were taken with a microscope, and the extents of glomerulosclerosis were evaluated based on the area of glomerular PAS staining. To evaluate the glomerulosclerosis score, 25 glomeruli were selected randomly from each mouse and scored based on the PAS-positive area as following: 0 point—normal glomeruli; 1 point—slight increase in thickness of the glomerular mesangial (PAS-positive area of <25%); 2 point—moderate segmental sclerosis (PAS-positive area of 25-50%); 3 point—severe segmental sclerosis (PAS-positive area of 50-75%); and 4 point—overall sclerosis (PAS-positive area ≥75%). The glomerulosclerosis scores were defined as the average score of 25 glomeruli.

### 7. Masson's Trichrome Staining and Renal Fibrosis Score

The renal fibrosis was estimated by using Masson's trichrome staining. The kidney taken from a mouse was fixed with 10% paraformaldehyde, dehydrated, and embedded in paraffin, and then was sliced into sections of 3 μm thick. The sections were stained with Weigert's iron hematoxylin working solution for 10 minutes and rinsed with distilled water. Thereafter, it was stained with Biebrich scarlet-acid fuchsin solution for 15 minutes, rinsed with distilled water, sliced, and stained with phosphomolybdic acid-phosphotungstic acid solution for 15 minutes. Finally, the sections were transferred into an aniline blue solution and stained for 5-10 minutes. Images of renal cortex were taken from each mouse randomly. The ratio of Masson's trichrome positive staining area/total renal cortex area was calculated by using the ImageJ version 1.44 image analysis software.

### 8. ELISA

The concentration of CCL7 was analyzed by using a human CCL7 quantikine ELISA kit (R&D systems) according to the instruction of the manufacturer.

### 9. Statistical Analysis

Results were presented as mean±standard derivation (SD). The statistical analysis was done by unpaired Student t test or ANOVA followed by Scheffe multiple comparison post-hoc test. Data was analyzed by using an SPSS software (Version 14; SPSS, Chicago, IL, USA). P-value of <0.05 was considered as statistically significant.

### Example 1: Plasma CCL7 Concentration in a Patient with Diabetic Kidney Disease Increased

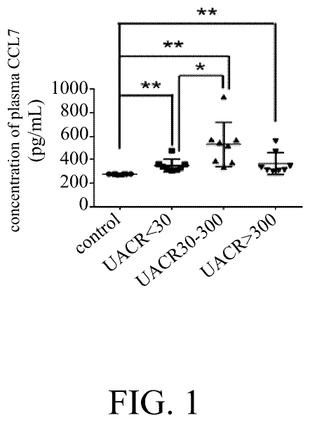

Subjects with and without diabetes were recruited, among which the subject having one of following cases was excluded: cancer or malignant disease recorded within recent 90 days, accompanied with infection, trauma or surgery. Plasma samples were collected from the subjects with and without diabetes, wherein the subjects with diabetes were further assigned to three subgroups: the subjects having a urinary albumin-creatinine ratio (UACR) less than 30 mg/g, the subjects having a UACR between 30 and 300 mg/g, and the subjects having a UACR greater than 300 mg/g. The concentration of CCL7 in each plasma sample was determined by ELISA, and the results were shown in FIG. 1 (n=8 per group, *P<0.05, **P<0.01).

As shown in FIG. 1, the subjects with diabetes had higher plasma CCL7 concentrations as compared to the subjects without diabetes, regardless of subgroup to which the urinary albumin-creatinine ratio (UACR) value belonged. In other words, the plasma CCL7 concentration in a subject having or in the risk of a diabetic kidney disease would increase. The aforementioned results showed that CCL7 was involved in the relevant mechanism of a diabetic kidney disease.

### Example 2: Inhibition of CCL7 Reduces Human Renal Proximal Tubular Epithelial Cells Inflammation and Fibrosis Induced by High Level of Glucose

To simulate the high blood glucose condition in a subject with diabetes, the CCL7 expression in human renal proximal tubular epithelial cells was stimulated and induced with high level of glucose in the example, and the expression of inflammatory/fibrosis protein, such as TNF-α, IL-6, IL-1β and TGF-β, in the epithelial cells induced by high level of glucose was determined.

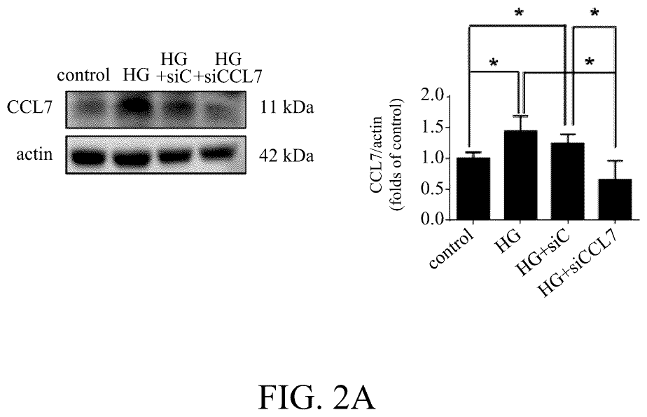

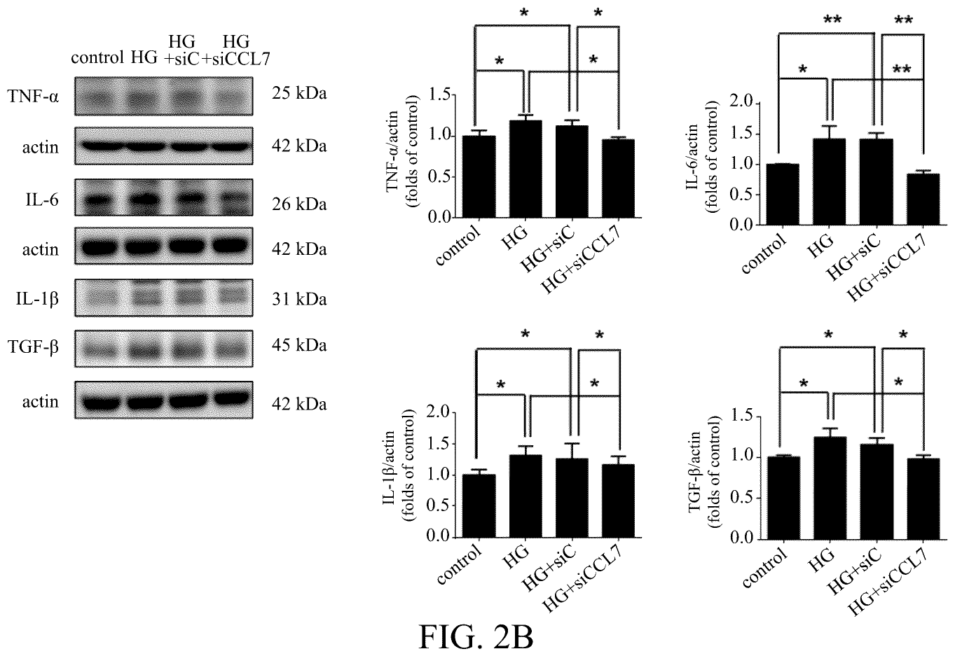

The experiments were carried out as described above in the Materials and methods 1 and 2, in brief, human renal proximal tubular epithelial cells were taken and assigned to four groups: a control group, an HG group, an HG+siC group and an HG+siCCL7 group, wherein the control group was cultured with DMEM, the HG group was cultured in DMEM containing 25 mM glucose (high glucose), and the HG+siC and the HG+siCCL7 groups were transfected with an siRNA control and a CCL7 siRNA, respectively, prior to culture in a high glucose medium. After being cultured for 2 days, proteins were extracted from the cells in each group and co-cultured with antibodies against CCL7, TNF-α, IL-6, IL-1β and TGF-β to determine the expression of CCL7, TNF-α, IL-6, IL-1β and TGF-β in the cells in each group. Finally, the expression level of each protein was normalized to the expression level of actin, and the results were shown in FIGS. 2A and 2B, respectively (n=3; *P<0.05, **P<0.01).

As shown in FIG. 2A, the expression level of CCL7 protein in the cells induced with high level of glucose in the HG group increased obviously, as compared with the control group. While reducing gene expression with CCL7 siRNA prior to the induction with high level of glucose could reduce the high glucose-caused CCL7 expression effectively. The aforementioned results showed that CCL7 siRNA surely can inhibit the expression of CCL7 effectively.

As shown in FIG. 2B, the expression level of inflammatory/fibrosis protein, such as TNF-α, IL-6, IL-1β and TGF-β, in the cells induced with high level of glucose in the HG group increased obviously, as compared with the control group. While reducing gene expression with CCL7 siRNA prior to the induction with high level of glucose could reduce the high glucose-caused inflammatory/fibrosis protein expression effectively. The aforementioned results showed that the suppression of CCL7 expression indeed can reduce the kidney inflammation and/or renal fibrosis caused by high level of glucose.

### Example 3: Inhibition of CCL7 could Improve the Proteinuria and Reduce Renal Hypertrophy in Mice Having Diabetic Kidney Disease

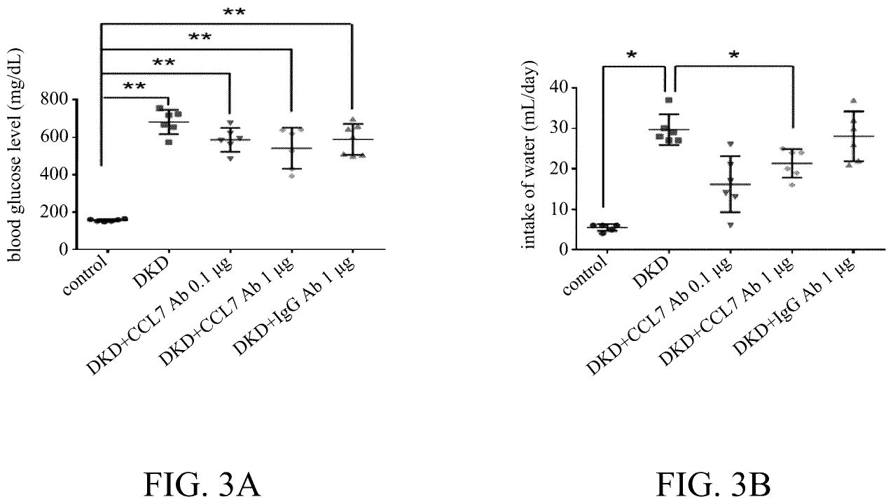

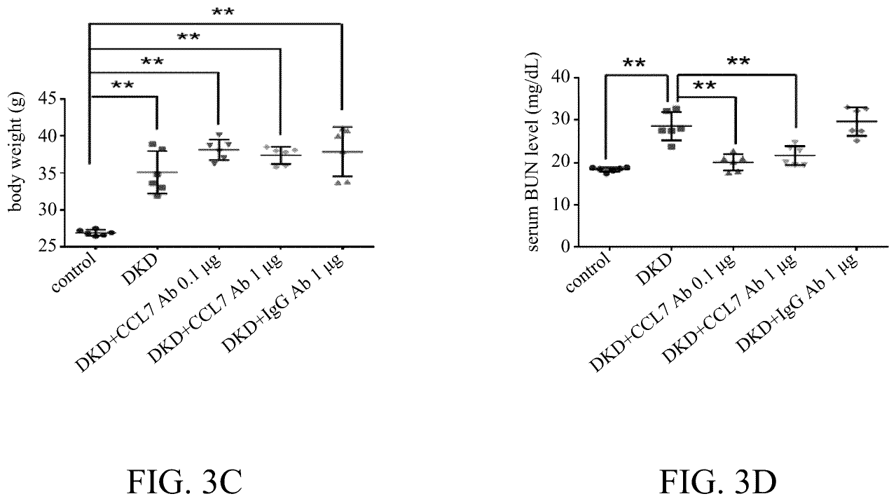

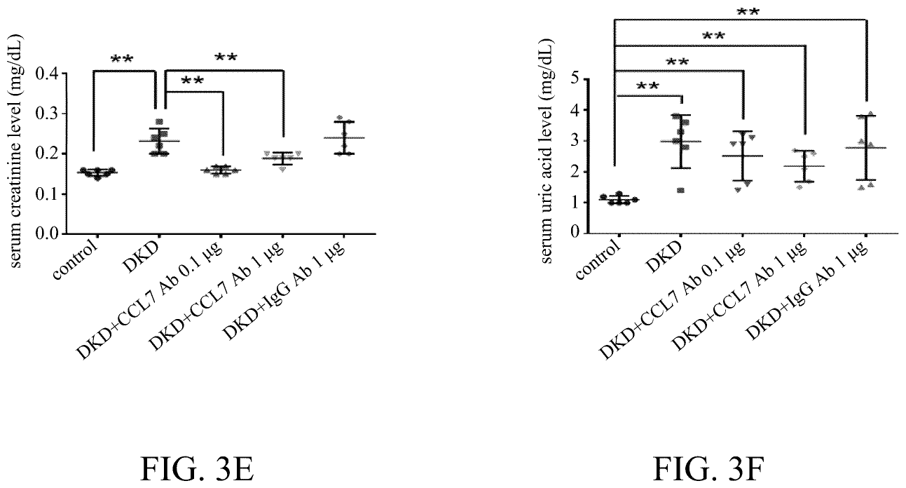

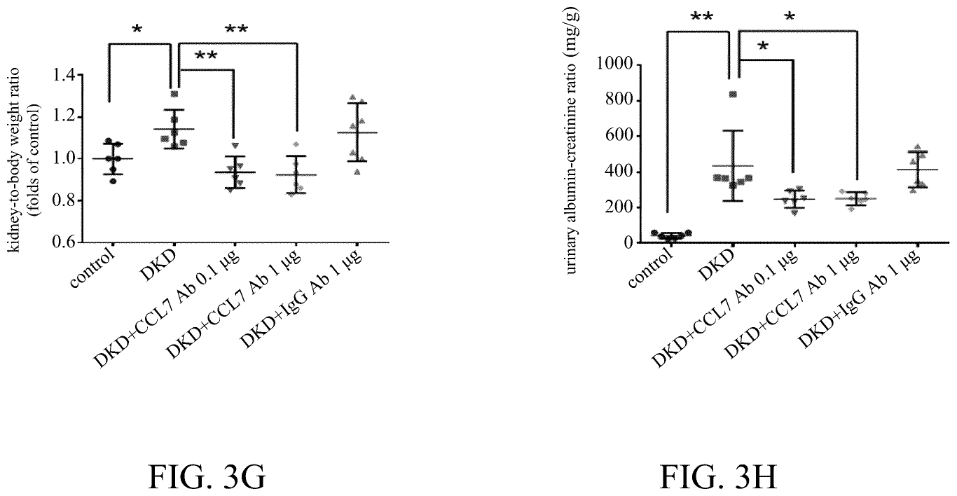

The experiment was carried out as described above in the Materials and methods 3 and 4, in brief, the 6-week aged male BKS.Cg-Dock7^{m}+/+ Lepr^{db} mice prepared were assigned randomly to four groups: a DKD group (mice having diabetic kidney disease), a DKD+CCL7 Ab 0.1 μg group (mice having diabetic kidney disease treated with 0.1 μg of CCL7 antibody), a DKD+CCL7 Ab 1 μg group (mice having diabetic kidney disease treated with 1 μg of CCL7 antibody), a DKD+IgG Ab 1 μg group (mice having diabetic kidney disease treated with 1 μg of IgG antibody); and the wild type littermates thereof (non-diabetes mice) were used as a control group. Thereafter, the mice were measured for the fasting blood glucose level with a glucometer, and were subjected to blood drawing for measurement of levels of serum blood urea nitrogen (BUN), serum creatinine (Cre) and serum uric acid. In addition, mice were places in metabolism cages to collect urine over 24 hours for determination of the urinary albumin-creatinine ratio, and at the meanwhile, the mice in each group were monitored for water intakes and body weights. Finally, the mice were sacrificed, and kidneys were taken from the mice for measurement of weights to calculate the kidney-to-body weight ratio. Results were shown in FIGS. 3A-3H (n=6; *P<0.05, **P<0.01).

As shown FIGS. 3A, 3C and 3F, the levels of fasting blood glucose, body weight and uric acid of mice having diabetic kidney disease were higher, and the treatment with CCL7 antibody had no effect on the values of levels of fasting blood glucose, body weight and uric acid, as compared with the control group. In addition, as shown in FIGS. 3B, 3D, 3E, 3G and 3H, there are significant increases in water intakes, serum blood urea nitrogen (BUN) levels, serum creatinine (Cre) levels, kidney-to-body weight ratios, and urinary albumin-creatinine ratios in the untreated mice having a diabetic kidney disease, as compared to the control group. But after the treatment with CCL7 antibody, the mice having diabetic kidney disease had obviously reduced water intakes, serum blood urea nitrogen (BUN) levels, serum creatinine (Cre) levels, kidney-to-body weight ratios and urinary albumin-creatinine ratios, even reduced to be similar to those of the control group. The aforementioned results showed that the inhibition of CCL7 achieved the effects of reducing dysphoric symptoms, improving each biochemical value and reducing the kidney-to-body weight ratio in the mice having diabetic kidney disease, and thus make a contribution to protection of renal functions.

### Example 4: Inhibition of CCL7 could Decrease Glomerular Hypertrophy, Glomerulosclerosis and Fibrosis in Mice Having Diabetic Kidney Disease

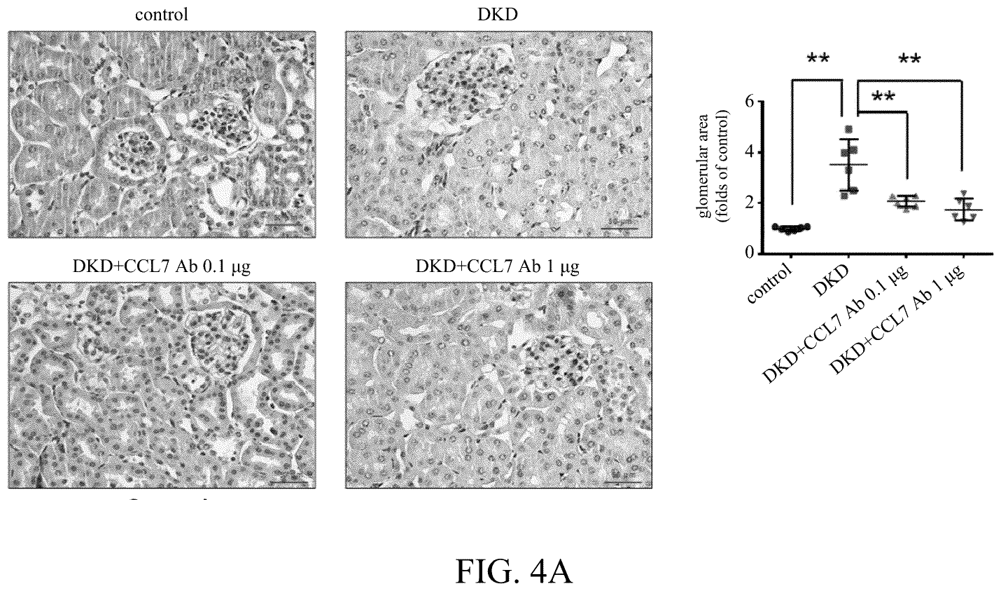

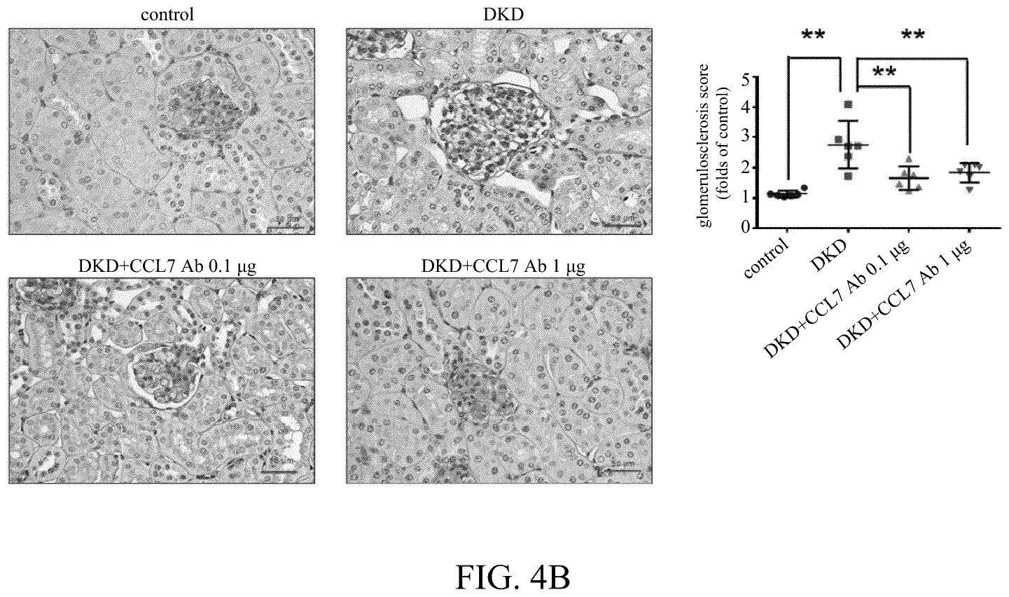

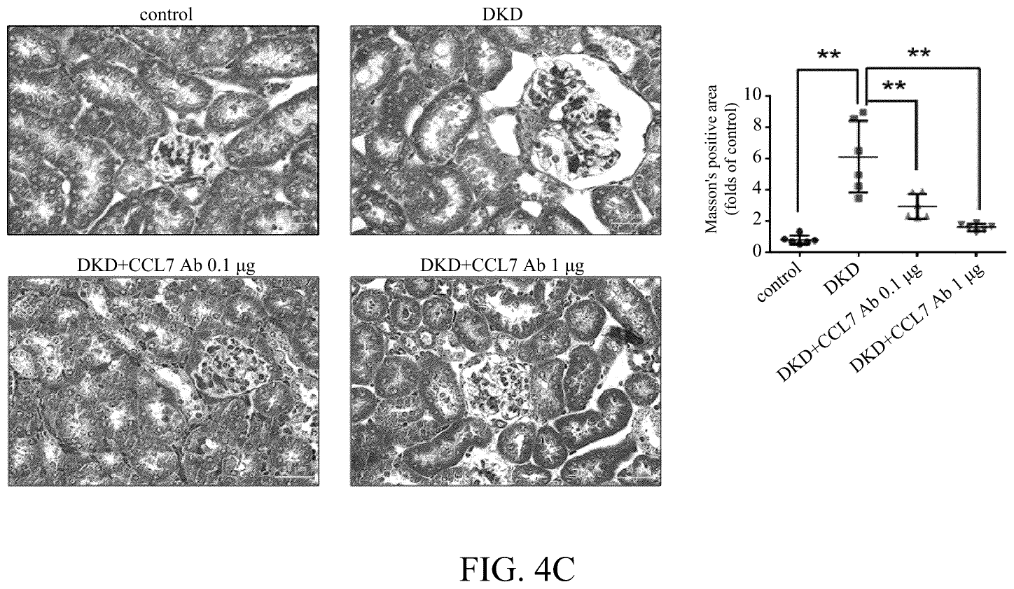

To evaluate the histological change associated with a diabetic kidney disease, HE staining, PAS staining, Masson's trichrome staining and quantifications were performed on the kidneys taken from the mice in each group, respectively, as described above in the Materials and methods 5-7, and the results were shown in FIG. 4A (HE staining), 4B (PAS staining) and FIG. 4C (Masson's trichrome staining) (n=6; *P<0.05, **P<0.01).

As shown in FIG. 4A, mice with diabetic kidney disease had significantly increased glomerular area, and after the treatment with CCL7 antibody, the glomerulus had slightly changed appearance and reduced Bowman space, increased glomerular cell content and reduced tubular lumen, as well as significantly reduced glomerular area, as compared with the control group. The aforementioned results showed that the inhibition of CCL7 indeed can achieve the effect of preventing and/or treating glomerular hypertrophy in mice having diabetic kidney disease.

As shown in FIG. 4B, mice with diabetic kidney disease had significantly increased glomerulosclerosis score, and after the treatment with CCL7 antibody, the mice with diabetic kidney disease had significantly reduced expansion of glomerular mesangial and accumulation of extracellular matrix as well as significantly reduced glomerulosclerosis score, as compared with the control group. The aforementioned results showed that the inhibition of CCL7 indeed can achieve the effect of preventing and/or treating glomerulosclerosis in mice having diabetic kidney disease.

As shown in FIG. 4C, mice with diabetic kidney disease had significantly increased Masson-positive area (i.e., collagen-positive area), and had significantly reduced Masson-positive area after the treatment with CCL7 antibody, as compared with the control group. The aforementioned results showed that the inhibition of CCL7 can achieve the effect of preventing and/or treating glomerular fibrosis in mice having diabetic kidney disease.

The experiment aforementioned results has confirmed that the inhibition of CCL7 could suppress the expression of the inflammatory factors and/or fibrous protein in epithelial cells effectively, reduce the dysphoric symptom in mice having diabetic kidney disease, ameliorating each biochemical value, reducing the kidney-to-body weight ratio, and decreasing glomerular hypertrophy, glomerulosclerosis and fibrosis, thereby protecting the renal functions, achieving the effect of preventing and/or treating diabetic kidney disease Accordingly, it should be understood by one with ordinary skill in the art, the administration of any CCL7 antagonist (any antibody, RNA agent, small molecular protein, compound, product from human body, natural product, natural extract, etc. capable of inhibiting the CCL7 activity) to a subject can achieve an inhibitory effect on CCL7, thereby preventing and/or treating diabetic kidney disease.

Even though numerous characteristics and advantages of the present disclosure have been set forth in the foregoing description, together with details of the structure and features of the invention, the invention is illustrative only. Changes may be made in the details, especially in matters of shape, size, and arrangement of parts within the principles of the invention to the full extent indicated by the broad general meaning of the terms in which the appended claims are expressed.

What is claimed is:

1. A method for preventing or treating a diabetic kidney disease in a subject in need thereof, comprising administering an effective amount of a chemokine C-C motif ligand 7 (CCL7) antagonist and/or a pharmaceutically acceptable salt thereof to the subject to inhibit an activity of CCL7.

2. The method according to claim 1, wherein the CCL7 antagonist is an antibody or an aptamer against the CCL7 or a CCL7 receptor.

3. The method according to claim 1, wherein the CCL7 antagonist is at least one selected from the group consisting of a CCL7 neutralizing antibody, a CCL7 RNA interference agent, a CCL7 small molecular antagonist, a C-C chemokine receptor type 1 antagonist, a C-C chemokine receptor type 2 antagonist, a C-C chemokine receptor type 3 antagonist, and a C-C chemokine receptor type 5 antagonist.

4. The method according to claim 3, wherein the CCL7 antagonist is the CCL7 neutralizing antibody and/or the CCL7 RNA interference agent.

5. The method according to claim 1, wherein the administering protects renal functions, reduces proteinuria, reduces kidney hypertrophy, reduces kidney inflammation and/or reduces kidney fibrosis.

6. The method according to claim 1, wherein the CCL7 antagonist and/or a pharmaceutically acceptable salt thereof is administered orally, intraperitoneally, intravenously, intradermally, intramuscularly, subcutaneously, or transcutaneously.

7. The method according to claim 1, wherein the CCL7 antagonist and/or a pharmaceutically acceptable salt thereof is administered in combination with an angiotensin-converting-enzyme inhibitor and/or an angiotensin II receptor antagonist.

8. The method according to claim 1, wherein the diabetic kidney disease is accompanied with urinary albumin.

9. The method according to claim 1, wherein the diabetic kidney disease is caused by at least one disorder of diabetes, hypertension, and hyperlipidemia.

10. The method according to claim 9, wherein the diabetes is type 1 diabetes or type 2 diabetes.

11. The method according to claim 1, wherein the diabetic kidney disease comprises an increase in an expression of an inflammatory factor and/or a fibrous protein in kidney, an increase in a level of a serum blood urea nitrogen, an increase in a level of a serum creatinine, an increase in a kidney-to-body weight ratio, and/or an increase in a urinary albumin-creatinine ratio.

12. The method according to claim 1, wherein the effective amount of the CCL7 antagonist and/or a pharmaceutically acceptable salt thereof is about 0.01 μg/kg to about 100 mg/kg.
